(19) United States
(12) Patent Application Publication
(10) Pub. No.: US 2023/0117236 A1
(43) Pub. Date: Apr. 20, 2023

(54) PHARMACEUTICAL COMPOSITION FOR COUNTERACTING REPRODUCTION AND SPREAD OF INFLUENZA VIRUSES

(71) Applicant: FLAMINA HOLDING AG, Zug (CH)

(72) Inventor: Michael MAY, Zug (CH)

(73) Assignee: FLAMINA HOLDING AG, Zug (CH)

(21) Appl. No.: 17/503,888

(22) Filed: Oct. 18, 2021

(51) Int. Cl. A61K 33/20; A61K 9/00; A61K 47/12

(52) U.S. Cl. CPC A61K 33/20; A61K 9/0056; A61K 47/12

(57) ABSTRACT

A pharmaceutical composition that is orally applied and that counteracts reproduction and the spread of influenza viruses, in particular influenza viruses of the so-called covid species and their mutations, comprises a pharmaceutically acceptable carrier and at least a first virucidal component designed to unfold antiviral activity when activated, which first virucidal component is sodium chlorite that is dissolved in saliva when the pharmaceutical composition has been applied to a person.

The present invention relates to a pharmaceutical composition used for counteracting reproduction and spread of influenza viruses.

### BACKGROUND OF THE INVENTION

A pandemic of a viral human-threatening influenza is based on the massive reproduction of individual influenza viruses and their transmission between humans.

Influenza viruses or Orthomyxoviridae, which infect epithelial cells in the upper and lower respiratory tract of an individual, use mucosal enzymes to prepare for entering target cells via receptor-mediated endocytosis.

The respiratory tract, which first is contacted by influenza viruses, is protected against dust particles and pathogens by the filtering function of the mucosa and the saliva secreted by the mucosa. The so-called mucosal immunity, which is based on immunoglobulins such as IgA, is an essential part of the human defence system.

For supporting the immune system, for example in the case of a cold, sore throat or influenza, lozenges, pills, sprays, masks, and vaccinations with antibacterial or antiviral agents are in use.

Antiviral influenza drugs, including the neuraminidase-based oseltamir, zanamivir, and peramivir, seem to have strengthened the drug resistance of influenza viruses and thus their mutational propensity.

The development of genetically modified immune-resistant virus strains, further developed for viral therapy as carriers for vaccines to combat cancer, has led to increasingly resistant and rapidly mutating variants, possibly supporting the Corona pandemic.

According to Lauren Sompayrac, HOW PATHOGENIC VIRUSES THINK, Making Sense of Virology, Jones & Bartlett Learning 2013, viruses are pieces of RNA or DNA enclosed in a protective coat. Every virus must solve four common problems if it is to survive and to infect the human body.

The first problem every virus must solve is how to access and infect its target cells. There are four major pathways for infection which viruses have evolved to use. Viruses can be inhaled, viruses can be ingested, viruses can be passed from mother to child, and viruses can be acquired by intimate physical contact. Each pathway has its own unique set of host defence mechanisms. Once a virus enters its new host, it must locate cells in which it can reproduce efficiently, and the choice of entry point will determine which cells are available for infection. To be an appropriate target for viral infection, a cell must have receptors on its surface to which the virus can attach. In addition, the biosynthetic machinery within the cell must be compatible with the reproductive strategy used by the virus. The second problem every virus must solve is how to reproduce within its target cells. No human virus carries with it the machinery (e.g., the ribosomes) required to synthesize proteins, and no human virus can generate the energy needed to power the copying (replication) of its genetic information. Therefore, viruses must “hijack” some of the biosynthetic machinery of the cells they infect and turn those cells into factories that can make many new copies of the virus. The third problem every successful virus must solve is how to evade the host’s antiviral defences long enough either to infect another host, or to establish a latent or chronic infection within the original host - from which the virus can spread at a later time.

The fourth problem every virus must solve is how to be transmitted from one infected individual to another.

The host defences consist of the anatomic barriers, the innate immune system and the adaptive immune system.

Vaccination typically triggers the adaptive immune system. Indeed, it is the goal of most vaccination strategies to produce memory B cells and protective antibodies, which however get active only after target cells have been affected by the intruding viruses, which may already have caused damage or may have further been distributed to other individuals.

Further, vaccination may cause side effects, may not be accepted and may not be readily available when a pandemic arrives. Reports indicate an association between the development of myocarditis and the receipt of messenger RNA (mRNA) vaccines against coronavirus disease 2019 (Covid-19), but the frequency and severity of myocarditis after vaccination have not been extensively explored.

Typically, people who have been cured of an influencer infection, such as a corona infection, by means of their own immune system are better protected against new infestations than people that were protected by vaccination. Further, since vaccination is strongly rejected by a considerable part of the population, possibly also due to reports pointing to side effects, herd immunity is scarcely reached.

The above facts suggest that it is extremely difficult to terminate pandemics of mutating influenza viruses.

Further, since viruses are dependent on the biochemical machinery of human cells for their reproduction, it is difficult to create drugs that will harm viruses, but not human cells.

Still further it is known that vaccinated individuals may still pass on the influenza virus to further individuals or may even still experience harmful infection.

### SUMMARY OF THE INVENTION

Based on the foregoing, the object of the present invention is providing a pharmaceutical composition for counteracting the reproduction and spread of influenza viruses.

The pharmaceutical composition shall be producible at low cost and shall be applicable efficiently and safely.

Furthermore, the pharmaceutical composition shall have the desired effect on any influenza virus without having harmful side-effects on the human body.

Still further, the pharmaceutical composition shall be well accepted by individuals requiring protection.

The pharmaceutical composition shall provide protection by its own but shall also allow supplementing vaccination regimes that typically never reach the whole population.

Further, the pharmaceutical composition shall provide additional protection to individuals that have already been vaccinated, so that passing on of the virus to further individuals or infection due to an intensive attack of influenza viruses is more securely excluded.

The object of the invention is reached with a pharmaceutical composition according to claim 1.

### DESCRIPTION OF THE INVENTION

According to the invention, a pharmaceutical composition is provided, that is orally applied and that counteracts reproduction and the spread of influenza viruses, in particular influenza viruses of the so-called covid species and their mutations. The pharmaceutical composition comprises a pharmaceutically acceptable carrier and at least a first virucidal component designed to unfold antiviral activity when activated, which first virucidal component is sodium chlorite (NaClO2) that is dissolved in saliva when the pharmaceutical composition has been applied to a person and that liberates chlorin dioxide (ClO2) when being dissolved in an weak acidic fluid.

When taken by an individual, the pharmaceutical composition is dissolved by the saliva of the individual, thereby dissolving the carrier and releasing and also dissolving and activating the virucidal component.

In a preferred embodiment at least one acidic activator is provided in the that is designed to accelerate activation of the virucidal component when the pharmaceutical composition has been applied to a person and is dissolved in saliva.

The inventive solution is targeted to destroying the reproductive capacity of viruses before they have entered host cells, i.e., before the processes of endocytosis take place. More particularly the inventive solution is targeted to destroying the protective coat of the virus before it has reached the target cells.

The invention is based on the consideration that influenza viruses for reproduction must penetrate into the human body via its respiratory tract.

For their replication, viruses preferentially attack those cells of an individual which are present in a warm, moist, and nutrient-rich environment such as is particularly present in the mucous membrane protecting the respiratory tract.

On the way of influenza viruses into a host cell of a human body, saliva and mucous membrane form a barrier (problem one described above) to stop, capture and hold the influenza viruses that have entered.

If influenza viruses succeed to pass this barrier, then a majority of the reproduced influenza viruses appear in the mucosa and underlying cells.

The influenza viruses, which are then formed by reproduction preferably and primarily in the nasal, palatal and oral cavity regions, then leave the human carrier at least predominantly via the respiratory orifices or are exhaled thereby solving problem four described above.

Consequentially, the mucosal area is unique, and essential to the reproduction and transfer of influenza viruses and forms a barrier preventing influenza viruses from entering and leaving the human body.

However, as is known, this innate barrier can be weakened for other reasons and can become insufficient for successfully inhibiting passing and multiplication of influenza viruses.

Hence, based on the inventive solution the innate barrier is enforced to increase the problems of influenza viruses when trying to pass on the way in and out of the human body.

The pharmaceutical composition is orally applied and dissolves in the saliva of the oral cavity regions. When released, the virucidal component in the form of sodium chlorite, which is an inorganic sodium salt with chlorite as the counterion, is activated by saliva or in the preferred embodiment by saliva and the at least one acidic activator.

When sodium chlorite gets in contact with saliva or with saliva and the acidic activator then the sodium chlorite is decomposed and chlorine dioxide is set free, which is absorbed by the saliva.

Chlorine dioxide even in the smallest concentration is capable to destroy the capsid and possibly the outer lipid of the influenza viruses, which are essential for their survival. Thereby also the DNA of the influenza viruses is made subject to an irreparable denaturation. Hence, by this process components of the influenza viruses are damaged in such a way that the reproduction and mutation of the influenza viruses penetrating through the mouth and nose is prevented to a large extent before they can enter a host cell or target cells, thus preventing multiplication and interrupting a potentially pandemic transmission to other individuals.

Due to the limited amount of saliva available, the release of chlorine dioxide takes place in smallest dosages. Instead, a low dosage of chlorine dioxide remains present for an extended period of time. Sodium chlorite not dissolved in the mouth is then decomposed in the stomach of the individual under the influence of hydrochloric acid.

Without the additional acidic activator, the virucidal component is dissolved by the saliva which normally is slightly acidic. Due to the low level of acidity the process of splitting of chlorine dioxide is done over a longer time period during which protection against the intrusion of viruses is established.

This process of splitting off chlorine dioxide is accelerated and antiviral activity is increased by the additional acidic activator, which is preferably an citric acid or a malic acid or an ascorbic acid or a mixture of at least two components thereof.

According to Lianger Dong and Yong Li, Fate of Salmonella Typhimurium and Listeria monocytogenes on Whole Papaya during Storage and Antimicrobial Efficiency of Aqueous Chlorine Dioxide Generated with HCl, Malic Acid or Lactic Acid on Whole Papaya, Foods 2021, 10, 1871. https://doi.org/10.3390/foods10081871, aqueous chlorine dioxide is gained when mixing sodium chlorite with an acid, such as HCl, lactic acid or malic acid.

According to N. Hatanaka, et al., Chlorine, Chlorine dioxide is a more potent antiviral agent against SARS-CoV-2 than sodium hypochlorite, the Journal of Hospital Infection, Volume 118, pages 20-26, Dec. 1, 2021, treatment with 24 ppm of chlorine dioxide inactivated more than 99.99% SARS-CoV-2 within 10 seconds.

The pharmaceutical composition is recommended to be taken whenever the individual is exposed to a risk of infection, e.g., when commuting in a transport system or when joining a group of people. Consequently, the pharmaceutical composition is taken during relatively short time intervals so that the biological balance of the human body is not impaired. Typically, the pharmaceutical composition will only be taken during time periods, in which the risk of infection has been communicated by responsible authorities.

The pharmaceutical composition comprises a carrier in a form and consistence that may correspond to products well known and accepted on the market. The carrier, which is for example a drop, a candy, a chewing gum, or the like, is designed to release the virucidal component or the virucidal component and the acidic activator when being dissolved in the saliva or when being chewed. The carrier preferably unfolds a flavour, which significantly contributes to or dominates the flavour of the pharmaceutical composition.

Preferably, the carrier comprises at least one additional virucidal component, such as one or more antiviral herbs, which help fighting influenza viruses. Known antiviral herbs are Oregano, Sage, Basil, Fennel, Garlic, Lemon balm, Peppermint, Rosemary, Echinacea, Sambucus, Astragalus, Ginger, Ginseng, or Dandelion. Hence, with one or more of these herbs, antiviral activity can be increased and a preferred flavour of the pharmaceutical composition can be created.

The at least one supplemental acidic activator is an acid, such as citric acid or malic acid or ascorbic acid or a mixture of at least two components thereof. The effect of the acidic activator is preferably twofold, on the one hand activating the virucidal component and on the other hand further contributing to the flavour of the pharmaceutical composition so that it is well accepted by individuals. Further, the acidic activator may further be beneficial to the individual by supporting his or her health system.

In a preferred embodiment the first virucidal component is provided in the carrier for dissolving in the saliva with an amount suitable for gaining chlorine dioxide in the saliva with a concentration in the range of 2 ppm to 50 ppm, more preferably in the range of 20 ppm to 30 ppm.

If an acidic activator is present, then the first virucidal component and the acidic activator are provided in the carrier for dissolving in the saliva with an amount suitable for gaining chlorine dioxide in the saliva with a concentration in the range of 20 ppm to 50 ppm, more preferably in the range of 20 ppm to 30 ppm.

If no acidic activator is present, then the amount of the virucidal component is increased, e.g., by a factor of 2 to 10.

The virucidal component and the at least one acidic activator may be contained in the carrier in any suitable form.

The virucidal component is preferably contained in the carrier in the form of a powder or micro pellets or dissolved in a liquid or gelee. The powder or micro pellets may be molten or not molten.

The acidic activator as well is preferably contained in the carrier in the form of a powder or micro pellets or dissolved in a liquid or gelee. The powder or micro pellets may be molten or not molten.

The virucidal component or the virucidal component and the at least one acidic activator may be distributed evenly or unevenly, within the carrier. Hence, during production the virucidal component or the virucidal component and the at least one acidic activator may be provided as a powder and may be mixed with a not yet solidified carrier material. The virucidal component or the virucidal component and the at least one acidic activator may also be provided mixed with the carrier material only in distinct regions of the carrier.

In preferred embodiments, the carrier is encapsulated within a shell which provides a preferred flavour when the pharmaceutical composition is orally applied, thus increasing acceptance of the pharmaceutical composition.

The invention is further described with reference to the drawings, which show in

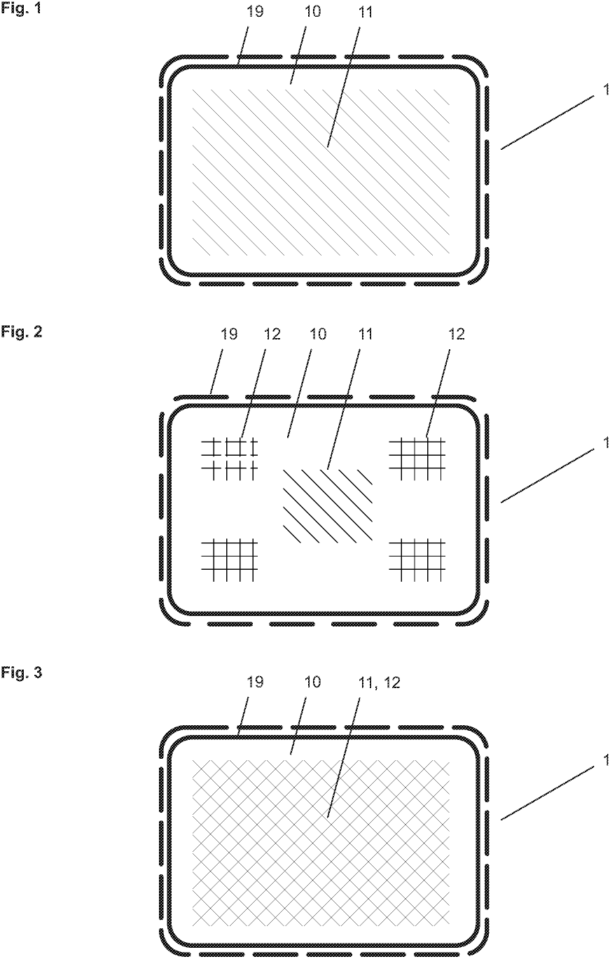

FIG. 1 a first embodiment of an inventive pharmaceutical composition 1;

FIG. 2 a second embodiment of an inventive pharmaceutical composition 1; and

FIG. 3 a third embodiment of an inventive pharmaceutical composition 1.

FIG. 1 shows a first embodiment of an inventive pharmaceutical composition 1, which comprises a carrier 10, e.g., made of candy material or chewing gum material. The carrier 10 is preferably encapsulated by a relatively thin shell 19 which provides a flavour preferred by the consumer.

The carrier 10 contains a virucidal component 11, which is evenly distribute within the carrier 10. Hence, while consuming the pharmaceutical composition 1, the slightly acidic saliva dissolves carrier 10 and the released virucidal component 11, i.e., the sodium chlorite. By this process chlorine dioxide is split off from the sodium chlorite and the acidic level in the saliva is increased up to the level within the predetermined range.

FIG. 2 shows the pharmaceutical composition 1 with a carrier 10 that contains a virucidal component 11 and one acidic activator 12 or more different acidic activators 12, which are located in different regions of the carrier 10. The regions with the virucidal component 11 and the acidic activator 12 may also overlap one another partially or completely. The at least one acidic activator 12 is preferably located in decentralised regions, while the virucidal component 11 is preferably located in a central region. Hence, while consuming the pharmaceutical composition 1, the acidic level in the saliva can build up by releasing the acidic activator 12 before the virucidal component 11 is set free. In this way the decomposition of the later dissolved virucidal component 11, i.e., of the sodium chlorite, to release chlorine dioxide is accelerated.

The earlier release of the acidic activator 12 has the further advantage that the taste of the pharmaceutical composition 1 will be dominated by the taste of the acidic activator 12, which may be a citric acid or a malic acid or an ascorbic acid or a mixture of at least two components thereof.

FIG. 3 shows the pharmaceutical composition 1 of FIG. 1 in a further preferred embodiment, in which the virucidal component 11 and the at least one acidic activator 12 are distributed, preferably evenly, within the carrier 10. In this embodiment, the virucidal component 11 and the at least one acidic activator 12 are in close contact, so that chlorine dioxide is released in minute quantities without delay.

Hence, by the application of the inventive pharmaceutical composition chlorine dioxide is released in small quantities without causing harm or disturbing distaste to the individual, while efficiently deactivating or neutralising influenza viruses.

1. A pharmaceutical composition that is orally applied and that counteracts reproduction and the spread of influenza viruses, comprising a pharmaceutically acceptable carrier and at least a first virucidal component designed to unfold antiviral activity when activated, which first virucidal component is sodium chlorite that is dissolved in saliva when the pharmaceutical composition has been applied to a person.

2. The pharmaceutical composition according to claim 1, comprising at least one acidic activator that is designed to activate the first virucidal component when the pharmaceutical composition has been applied to a person.

3. The pharmaceutical composition according to claim 1, wherein the at least one acidic activator is an citric acid or a malic acid or an ascorbic acid or a mixture of at least two components thereof.

4. The pharmaceutical composition according to claim 1, wherein the carrier is a drop, a candy, a chewing gum, or the like, which is designed to release the first virucidal component or the first virucidal component and the acidic activator when being dissolved in the saliva or when being chewed.

5. The pharmaceutical composition according to claim 1, wherein the first virucidal component is provided in the carrier for dissolving in the saliva with an amount suitable for gaining chlorine dioxide in the saliva with a concentration in the range of 2 ppm to 50 ppm.

6. The pharmaceutical composition according to claim 1, wherein the first virucidal component and the acidic activator are provided in the carrier for dissolving in the saliva with an amount suitable for gaining chlorine dioxide in the saliva with a concentration in the range of 2 ppm to 50 ppm.

7. The pharmaceutical composition according to claim 1, wherein
a) the first virucidal component is unevenly distributed or evenly distributed within the carrier; or
b) wherein the at least one acidic activator is unevenly distributed or evenly distributed within the carrier.

8. The pharmaceutical composition according to claim 1, wherein the first virucidal component is unevenly distributed or evenly distributed within the carrier and the at least one acidic activator is unevenly distributed or evenly distributed within the carrier.

9. The pharmaceutical composition according to claim 1, wherein the first virucidal component is contained in the carrier in the form of a powder or in the form of micro pellets.

10. The pharmaceutical composition according to claim 1, wherein the acidic activator is contained in the carrier in the form of a powder or micro pellets or wherein the acidic activator is contained in the carrier dissolved in a liquid.

11. The pharmaceutical composition according to claim 1, wherein at least a second virucidal component is provided in the carrier.

12. The pharmaceutical composition according to claim 1, wherein the carrier is covered by a shell with a specific flavour.
